(19) United States
(12) Patent Application Publication
(10) Pub. No.: US 2008/0107719 A1
(43) Pub. Date: May 8, 2008

(54) Transdermal drug delivery system

(76) Inventors: Sukhon Likitlersuang, Irvine, CA (US); Chin-Ming Chang, Tustin, CA (US); James Chang, Newport Beach, CA (US); Orest Olejnik, Coto De Caza, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/594,517

(22) Filed: Nov. 8, 2006

(51) Int. Cl. A61K 9/70; A61K 31/13; A61P 25/28

(52) U.S. Cl. 424/449; 514/579; 604/20; 514/661

(57) ABSTRACT

A transdermal drug delivery system includes a drug source, an adhesive for applying the system to a skin surface, a penetration enhancer for enhancing penetration of the drug into the skin surface, an energy source and a contact for conveying energy from said energy source through said adhesive and the penetrating enhancer.

Memantine HCl is served for the purpose of preserving visual function for glaucoma patients. The present memantine HCl formulation is designed for oral administration. Dosing titration instruction for therapy in order to achieve successful treatment is quite complicated; therefore, the patient compliance is a concern. Additionally, in order to maintain therapy more effectively with minimal side effects, memantine blood concentration should be kept constantly in therapeutic window.

Alpha-2B agonist is served for pain treatment. Compounds categorized as alpha-2B agonist are AGN 199981, 203818, and 201781. At development stage by Allergan, Inc., these compounds are designed for oral administration. Local pain treatment is in high patient demand due to acute treatment and lower systemic side effects. Additionally, in order to maintain therapy more effectively with minimal side effects, alpha-2B blood concentration should be kept constantly in therapeutic window.

The drawbacks of conventional orally drug therapy is known as systemic first pass metabolism through liver and the drastic changes in PH along gastrointestinal tract which affect drug bioavailability.

Transdermal drug delivery is effectively for controlling drug level in plasma and sustaining the drug therapeutic level in systemic circulation, no longer frequent drug intake and controlling undesirable side effects. The challenging aspects of this system are barrier property of skin to prevent the entry of drug molecule and enzyme metabolism of micro flora on skin surface. Therefore, the optimal physicochemical properties, biological properties and suitable permeation enhancer are factors that are taken into a consideration.

The potential possible rate-controlling factors of skin permeation are

### 1. Diffusion through the delivery device/formulation

### 2. Diffusion through the stratum corneum due to complexity of membrane and micro flora enzyme on skin membrane

### 3. Diffusion through the dermis into blood circulation.

### Therefore, the candidate drug passing through skin should have optimal lipophilicity and hydrophilicity in order to overcome all these possible factors.

Skin hydration state is the one of the most important factors in determining the rate of percutaneous absorption of a given solute. The level of hydration is ability of the stratum corneum to bind water. Skin penetration enhancer added into device formulation is responsible for keeping suitable skin hydration state in order to promote percutaneous drug absorption.

### SUMMARY OF THE INVENTION

A transdermal drug delivery system in accordance with the present invention generally includes a drug source, specifically, Memantine or Alpha-2B agonist along with an adhesive for applying the system to a skin surface.

A penetration enhancer is provided for enhancing penetration of the drug into a skin's surface and an energy source along with a means for conveying the energy from the energy source through the adhesive and penetration enhancer to further, in combination, enhance delivery of drug through the skin surface.

In one embodiment, the penetration enhancer is incorporated into the drug source and another embodiment the penetration enhancer is incorporated into the adhesive.

Still, a further embodiment of the present invention provides for a membrane, disposed between these drug source and adhesive with the penetration enhancer incorporated into the membrane.

In another embodiment of the present invention, the drug source is incorporated into the adhesive and may still further embodiment of the drug source is a reservoir.

More specifically, the system in accordance with the present invention includes a source of energy selected from a group consisting of electrical energy, ultrasonic energy, and heat energy. Further, the specific penetration enhancer may be menthone, Terpineol and Cineole, Geraniol, Thymol, pyrolidone ring derivatives, polysorbate, alkyl ether and poloxamer.

### BRIEF DESCRIPTION OF THE DRAWINGS

The advantages and features of the present invention will be better understood by the following description when considered in conjunction with the accompanying drawings, in which:

FIG. 1 is a plan view of a transdermal drug delivery system generally showing a drug source, a controller, an adhesive, an energy source and means for conveying energy from the energy source; and

FIGS. 2-5 are perspective cross sectional views of the drug source controller/membrane and adhesive shown in FIG. 1.

### DETAILED DESCRIPTION

The memantine HCl dosage recommended is ranged from 5 mg to 20 mg per day which are considered as small quantities over period of a day. Therefore, with a minimal dose delivered into blood circulation by passing skin barrier, it is quite a promising successful through transdermal drug delivery.

Memantine HCl molecular weight is 215.77, which is optimal for being a drug candidate in transdermal delivery system which requires molecular weight ranged between 100-500.

Once memantine partition and diffuse through membrane then partition into the underlying tissue, favorable log P o/w 3.28 of memantine indicates reasonable partitioning property which has optimal lipophilic and hydrophilic characteristics. The recommended log Po/w is ranged between 1-3.

The steady state flux of memantine through skin membrane is required to achieve the optimal and predicted drug therapeutic level in systemic circulation as following flick's law. Therefore, sink condition in one side and infinite dose of applied drug are condition required to reach steady state flux of memantine. The concentration gradient from applied site to sink condition side is proportional to solubility of memantine, 45 mg/ml considered as high solubility with suitable lipophilic characteristic due to adamantane structure.

With having high stability in both acidic and basic pH conditions, memantine shows the promising tolerability to acidic skin condition, which is one of the rate limiting steps of skin permeation.

The physiochemical properties of Memantine (AGN 192944) are shown in Table 1.

TABLE 1

| | Memantine HCl | Drug Candidate |
|---|---|---|
| API Name/ AGN Number | AGN 192944 | Property |
| Chemical Structure | 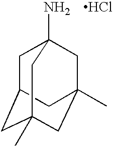 | Dose: Few mg range |
| Empirical Formular | C₁₂H₂₁N.HCL | |
| Molecular weight | 215.77 | 100-500 |
| Appearance | White odorless | N/A |
| | crystalline powder | |
| Morphology | Needle shape, white | N/A |
| | crystalline powder | |
| Melting point (° C.) | 290-295 | low M.P increase |
| | | drug penetration |
| Hygroscopicity | Slightly hygroscopic | N/A |
| | (<1.1% moisture | |
| | sorption at 5-95% RH) | |
| Pka | 10.53 | |
| Log P (Octanol/water) | 3.28 | Range 1-3 |
| Particle size (μm) | high variability among | N/A |
| (Mean ± S.D.) | lot-to-lot | |
| Stability in aqueous (PH1 | Stable | Stable |
| and 14) tested by 3% | | |
| hydrogen peroxide | | |
| Solubility in water | 45 mg/ml (24 hr) | Optimal |
| | 1% aqueous solution | |
| | (PH 5.5-6) | |
| Solubility (μg/ml) | Higher than 1.2 mg/ml | Optimal |
| in various PH medium | in PH 2-8 | |

The alpha-2B dosage is in few milligram ranges. Therefore, with a minimal dose delivered into blood circulation by passing skin barrier, it is quite a promising successful through transdermal drug delivery.

Alpha-2B agonist molecular weight is ranged 180-260, which is optimal for being a drug candidate in transdermal delivery system which requires molecular weight ranged between 100-500.

With favorable log P o/w of 1.5-3.28 of alpha-2B, it indicates quite promising partitioning property with optimal lipophilic and hydrophilic characteristics. The recommended log Po/w is ranged between 1-3.

The steady state flux of alpha-2B through skin membrane is required to achieve the optimal and predicted drug therapeutic level in systemic circulation as following flick's law. Therefore, sink condition in one side and infinite dose of applied drug are condition required to reach steady state flux of alpha-2B. The concentration gradient from applied site to sink condition side is proportional to solubility of alpha-2B, which has quite good solubility (1%-20% solution) and optimal lipophilic characteristic.

Additionally, in aspect of stability, alpha2-B having long half life in both acidic and basic conditions (PH1-11) indicates the tolerant to the acidic pH condition due to micro flora bacteria on skin which is one of the rate limiting steps of drug penetrating through skin.

The physiochemical properties of Alpha-2B agent (AGN 2038-18) are shown in Table 2.

TABLE 2

| API Name/ | | |
|---|---|---|
| AGN Number | AGN 203818 | Drug Candidate |
| Chemical Structure | 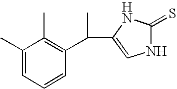 | Dosage:Few miligrams |
| Empirical Formular | C₁₃H₁₆N₂S.HCL | |
| Molecular weight | 232.35 | 100-500 |
| Melting point (° C.) | 270.31 | Low M.P. increases |
| | | drug penetration. |
| Pka | 11.26 | |
| Log P | 2.9-3.12 | Range 1-3 |
| (Octanol/water) | | |
| t1/2 (days) PH1 | 51 | Long t1/2 |
| t1/2 (days) PH11 | 57 | Long t1/2 |
| Solubility in water | 24.5 μg/m (>24 hr) | Optimal |
| Solubility in PG | >5.1 | Soluble in cosolvent |
| (mg/ml) | | |
| Solubility (μg/ml) in | 19.3-27.5 μg/ml in PH1-11 | Optimal |
| various PH medium | | |

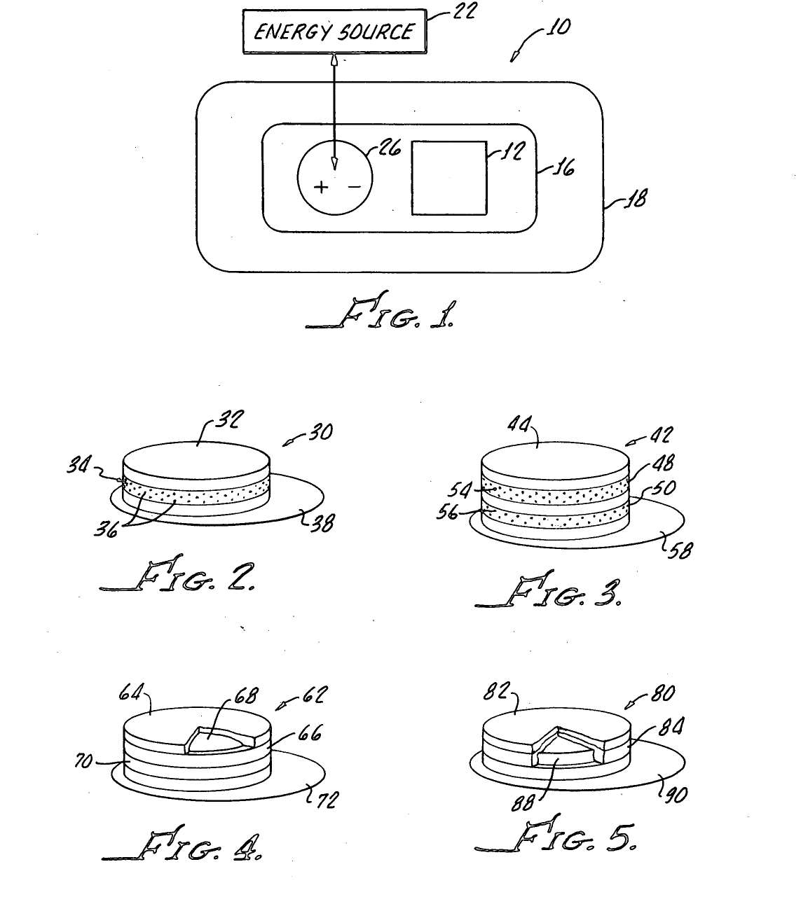

Turning now to FIG. 1, there is shown a transdermal drug delivery system 10 in accordance with the present invention generally including a source of memantine or Alpha-2B agonist which may include a reservoir 12, a controlling membrane 16 along with an adhesive 18 for application of the system 10 to a skin's surface (not shown).

As will be hereinafter described in greater detail, an energy source 22 is provided along with a contact 26 which provides a means for conveying energy from the energy source 22 through the reservoir 12 membrane 16 and adhesive 18.

The percutaneous penetration in accordance with the present invention incorporates both chemical and physical enhancement, the chemical aspects utilizing a penetration enhancer and the physical aspects utilizing an energy source.

Various layers of drug source 12 membranes and adhesives may be utilized and illustrated in FIGS. 2-5.

In FIG. 2, there is shown a layer configuration 30 utilizing a backing 32, an adhesive 34 incorporating a drug 36 along with a removable liner 38.

FIG. 3 illustrates an alternate layer configuration 42 including a backing 44, two adhesive layers 48, 50 with embedded drug 54, 56 and a removable liner 58.

FIG. 4 illustrates still another layer configuration embodiment 62 which includes a backing 64, a membrane 66 along with a drug reservoir 68, adhesive 70, and liner 72.

Yet another embodiment of a layer configuration 80 is illustrated in FIG. 5, which includes a backing 82, adhesive 84, a drug reservoir 88, and liner 90. All of these configurations may be utilized in a combination of enhancing drug delivery by utilizing both a penetration enhancer and an energy source.

### 1. Summary of the Chemical Enhancement Portion of the Present Invention

(a) Drug reservoir: The Memantine or Alpha-2B agent drug layer involves formulation of a semisolid cream or gel in which enhancer and cosolvent, emulsifier and/or surfactant is present as a solution or finely dispersed suspension along with the drug or enhancer incorporated in adhesive membrane in order to control drug release rate. On weight basis, drug is 1-10%, penetration enhancer is 1-10-% and the rest are mixture of vehicle and alcoholic cosolvent. The alcoholic cosolvents enhance drug solubility in drug mixtures in order to promote passive diffusion through skin membrane. Additionally, cosolvents promote cooling effect, function of permeation enhancer and touch-dry effects to the area of skin to which drug delivery system is applied.

(b) Backings: Backings is in the form of mono-layer, multi-layer, multi-laminate, non-woven, woven or metallic.

(c) Membrane used for controlling drug release from a device.

(d) Adhesive: Adhesive is used for attaching a device to skin membrane.

(e) Liner: A liner serves as a protectant or carrier for an adhesive film and easily be removed.

This present delivery is preferable to administer to both human and non-human animal. The optimal dosage of memantine will be adjusted to equivalently utilize for pain treatment. Therefore, both amount of memantine and penetration enhancer will be adjusted as influentially by the desired effect.

### Penetration Enhancer Used

Percentage recommended range of penetration enhancer is ranged 1-10%. Due to the fact that desirable attributes of penetration enhancers should be pharmacological inert, minimal toxic level, less harmful to skin, exert reversible effect of skin as material is removed from skin, chemical and physically compatible with memantine and formulate into lotions, suspension, gels, ointment, creams, aerosols, preferable permeation enhancer used in memantine transdermal system are:

1. Terpenes consists of acyclic, monocyclic, bicyclic and sesquiterpene, which have hydrocarbon terpene responsible for penetration enhancing lipophilic part of drug and oxygen part of terpene responsible for hydrophilic part of the drug. The promising enhancers are menthone, Terpineol and Cineole, Geraniol and Thymol with log P ranging from 2-3, which are close to log P of memantine HCl. Since terpenes are extracted from fruit and flowers and used as fragrances and flavoring agents, the safety and toxicity are not concerns.

2. All pyrolidone ring derivatives especially pyrolidone containing alkyl group at 1-position and 3-position. The pyrolidone derivative has profound affected to enhance protonized memantine to penetrate through skin. Example: N-methylpyrrolidone LD50 (skin) in rat is 7 g/KG bodyweight.

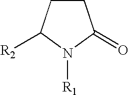

R₁ and R₂ are substitution groups.

3. Nonionic surfactant includes polysorbate, alkyl ether and poloxamer.

Example: Polysorbate80 has no adverse effect as skin is contacting. Additionally, polysorbate80 LD50 (Rat-Oral) is 34.5 ml/kg bodyweight; therefore, non-ionic surfactant is considered as safe materials used for skin with low irritation and toxicity.

### Cosolvent Used

Examples of alcoholic cosolvent used are alcohol, propylene glycol and polyethylene glycol 400. Cosolvent increase skin hydration which promote drug contacting into tissue for absorption.

### Examples of Drug Reservoir

EXAMPLE 1

| Component | Amount |
|---|---|
| Memantine | 5% |
| 1-Methyl-2-pyrrolidone | 2% |
| Aqueous alcoholic mixtures | to 100 ml |

EXAMPLE 2

| Component | Amount |
|---|---|
| Memantine | 5% |
| Menthone | 2% |
| Aqueous alcoholic mixtures | to 100 ml |

EXAMPLE 3

| Component | Amount |
|---|---|
| Memantine | 5% |
| Polysorbate80 | 2% |
| Aqueous alcoholic mixtures | to 100 ml |

EXAMPLE 4

| Component | Amount |
|---|---|
| Memantine | 5% |
| Menthone | 1% |
| Polysorbate80 | 1% |
| Aqueous alcoholic mixtures | to 100 ml |

EXAMPLE 5

| Component | Amount |
|---|---|
| Memantine | 5% |
| 1-Methyl-2-pyrrolidone | 1% |
| Polysorbate80 | 1% |
| Aqueous alcoholic mixtures | to 100 ml |

EXAMPLE 6

| Component | Amount |
|---|---|
| AGN 203818 | 5% |
| 1-Methyl-2-pyrrolidone | 2% |
| Aqueous alcoholic mixtures | to 100 ml |

EXAMPLE 7

| Component | Amount |
|---|---|
| AGN 203818 | 5% |
| Menthone | 2% |
| Aqueous alcoholic mixtures | to 100 ml |

EXAMPLE 8

| Component | Amount |
|---|---|
| AGN 203818 | 5% |
| Polysorbate80 | 2% |
| Aqueous alcoholic mixtures | to 100 ml |

EXAMPLE 9

| Component | Amount |
|---|---|
| AGN 203818 | 5% |
| Menthone | 1% |
| Polysorbate80 | 1% |
| Aqueous alcoholic mixtures | to 100 ml |

EXAMPLE 10

| Component | Amount |
|---|---|
| AGN 203818 | 5% |
| 1-Methyl-2-pyrrolidone | 1% |
| Polysorbate 80 | 1% |
| Aqueous alcoholic mixtures | to 100 ml |

2. Physical Penetration Enhancement

Memantine HCl skin penetration employs energy sources such as ultrasound, heat, electrical current and modifies adhesive part to remove surface skin in order to delivery memantine into blood circulation more efficiently, controllably and precisely.

To energize the delivery pushing drug into blood stream, energy sources 22 such as ultrasound, electrical current or heat current are employed. The delivery is composed of one of the energy sources 22 as mentioned, the contact 26, drug reservoir 12 and adhesive 18. A mild electrical current promotes the migration of ion or charged molecules through the skin in a conventional manner. The positively and/or negatively charged drug is introduced from charged electrode. Additionally, ultrasound and electric pulse improves drug transport through skin by altering lipid component of skin structure; therefore, skin penetration enhancement of drug occurs.

The use of ultrasound as the energy source would require the contact 26 to include a coupling medium. Heat may be applied directly to the contact.

By using synergistic effect of chemical enhancers and external energy sources, memantine HCl or Alpha-2B agents can be penetrated into skin efficiently and controllably. Chemical enhancers promote hydration state of skin in order to facilitate skin enhancement and energy sources (ex. ultrasound, heat and/or electrical current) affect the skin structure in order to facilitate drug to be absorbed precisely and efficiently. Preferable permeation enhancers used in memantine transdermal system are:

1. Terpenes consists of acyclic, monocyclic, bicyclic and sesquiterpene, which have hydrocarbon terpene responsible for penetration enhancing lipophilic part of memantine and oxygen part of terpene responsible for hydrophilic part of the memantine. The promising enhancers are menthone, Terpineol and Cineole, Geraniol and Thymol with log P ranging from 2-3, which are close to log P of memantine HCl. Since terpenes are extracted from fruit and flowers and used as fragrances and flavoring agents, the safety and toxicity is not a concern.

2. All pyrolidone ring derivatives especially pyrolidone containing alkyl group at 1-position and 3-position. The pyrolidone derivative has profound affected to enhance protonized memantine to penetrate through skin. Example: N-methylpyrrolidone LD50 (skin) in rat is 7 g/KG bodyweight.

3. Nonionic surfactant includes polysorbate, alkyl ether and poloxamer.

### 2. Summary of Physical Enhancement

(a) Drug reservoir: Memantine or Alpha-2B agent drug layer involves formulation of a semisolid cream or gel in which enhancer and cosolvent, emulsifier and/or surfactant is present as a solution or finely dispersed suspension along with the drug or enhancer incorporated in adhesive membrane in order to control drug release rate. On weight basis, drug is 1-5%, penetration enhancer is 1-10-% and the rest are mixture of vehicle and alcoholic cosolvent. The alcoholic cosolvents enhance memantine solubility in drug mixtures in order to promote passive diffusion through skin membrane. Additionally, cosolvents promote cooling and touch-dry effects to the area of skin to which drug delivery system is applied.

### Examples of Drug Reservoir

EXAMPLE 1

| Component | Amount |
|---|---|
| Memantine or AGN 203818 | 5% |
| 1-Methyl-2-pyrrolidone | 2% |
| Aqueous alcoholic mixtures | to 100 ml |

EXAMPLE 2

| Component | Amount |
|---|---|
| Memantine or AGN 203818 | 5% |
| Menthone | 2% |
| Aqueous alcoholic mixtures | to 100 ml |

EXAMPLE 3

| Component | Amount |
|---|---|
| Memantine or AGN 203818 | 5% |
| Polysorbate80 | 2% |
| Aqueous alcoholic mixtures | to 100 ml |

EXAMPLE 4

| Component | Amount |
|---|---|
| Memantine or AGN 203818 | 5% |
| Menthone | 1% |
| Polysorbate80 | 1% |
| Aqueous alcoholic mixtures | to 100 ml |

EXAMPLE 5

| Component | Amount |
|---|---|
| Memantine or AGN 203818 | 5% |
| 1-Methyl-2-pyrrolidone | 1% |
| Polysorbate80 | 1% |
| Aqueous alcoholic mixtures | to 100 ml |

(b) Backing: Backing is in the form of mono-layer, multi-layer, multi-laminate, non-woven, woven or metallic.

(c) Membrane

(d) Adhesive

(e) Liner

(f) Energy source located on the top of controller: ultrasound, heat, and/or electrical current

Although there has been hereinabove described a specific transdermal drug delivery system in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1. A transdermal drug delivery system comprising:
a source of memantine;
an adhesive for applying the system to a skin surface;
a penetration enhancer for enhancing penetration of memantine into said skin surface;
an energy source; and
means for conveying energy from said energy source through said adhesive and said penetration enhancer to the skin surface.

2. The system according to claim 1 wherein said penetration enhancer is incorporated in said source of memantine.

3. The system according to claim 1 wherein said penetration enhancer is incorporated in said adhesive.

4. The system according to claim 1 further comprising a membrane, disposed between said source of memantine and said adhesive and said penetration enhancer is incorporated into said membrane.

5. The system according to claim 1 wherein said source of memantine is incorporated into said adhesive.

6. The system according to claim 1 wherein said source of memantine is a reservoir.

7. The system according to any one of claims 1-6 wherein said source of energy is selected from a group consisting of electrical energy, ultrasound energy, and heat energy.

8. The system according to claim 7 wherein said penetration enhancer is selected from a group consisting of menthone, Terpineol and Cineole, Geraniol, Thymol, pyrolidone ring derivatives polysorbate, alkyl ether and poloxamer.

9. A transdermal drug delivery system comprising:
a source of Alpha-2B agonist;
an adhesive for applying the system to a skin surface;
a penetration enhancer for enhancing penetration of Alpha-2B agonist into said skin surface;
an energy source; and
means for conveying energy from said energy source through said adhesive and said penetration enhancer to its skin surface.

10. The system according to claim 9 wherein said penetration enhancer is incorporated in said source of Alpha-2B.

11. The system according to claim 9 wherein said penetration enhancer is incorporated in said adhesive.

12. The system according to claim 9 further comprising a membrane, disposed between said source of Alpha-2B agonist and said adhesive and said penetration enhancer is incorporated into said membrane.

13. The system according to claim 9 wherein said source of Alpha-2B agonist is incorporated into said adhesive.

14. The system according to claim 9 wherein said source of Alpha-2B agonist is a reservoir.

15. The system according to any one of claims 9-14 wherein said source of energy is selected from a group consisting of electrical energy, ultrasound energy, and heat energy.

16. The system according to claim 15 wherein said penetration enhancer is selected from a group consisting of menthone, Terpineol and Cineole, Geraniol, Thymol, pyrolidone ring derivatives polysorbate, alkyl ether and poloxamer.
